(19) United States
(12) Patent Application Publication
(10) Pub. No.: US 2012/0289590 A1
(43) Pub. Date: Nov. 15, 2012

(54) HAIR COSMETIC COMPOSITION

(76) Inventors: Gerry Ritterman, Chappaqua, NY (US); Doug Rosen, Chappaqua, NY (US); Mort Westman, Oak Brook, IL (US)

(21) Appl. No.: 13/105,267

(22) Filed: May 11, 2011

(51) Int. Cl. A61K 31/355; A61P 17/10; A61P 17/00; A61P 31/04

(52) U.S. Cl. 514/458

(57) ABSTRACT

A hair cosmetic composition including an exfoliating agent, an antioxidant, and an antibacterial agent. The hair cosmetic composition is noncomedogenic and nonacnegenic.

### BACKGROUND OF THE INVENTION

1. Field of the Invention

The present invention is related to a non-comodegenic hair cosmetic composition.

2. Description of Related Art

Hair cosmetic compositions are widely used and offer a number of benefits to users. These cosmetic compositions are widely available in various forms including gels, liquids, foams, and lotions. Unfortunately, it is widely known that the use of these cosmetic compositions can cause acne.

There is a need for a hair cosmetic composition that will impart the beneficial aspects of the composition to the hair, while reducing the incidence of acne.

### SUMMARY OF THE INVENTION

The present disclosure provides a non-comedogenic hair cosmetic composition. The present disclosure further provides a hair cosmetic composition that includes an exfoliating agent in an amount of 0.1% to 10%, an antioxidant in an amount of 0.01% to 5.0%, and an antibacterial agent in an amount of 0.05% to 2.5%, based on the total weight of the hair cosmetic composition, in which the hair cosmetic composition is noncomedogenic and nonacnegenic.

The present disclosure also provides a method of making a hair cosmetic composition. The method comprises the step of forming a first solution by mixing at an ambient temperature, in the following order, a non-ionic surfactant, a fragrance, a silicone fluid, a modified phospholipid, an antibacterial agent, an antioxidant, and a quaternary ammonium chloride. The method further comprises the step of forming a second solution by adding a cationic polymer to a quantity of water at an ambient temperature with ample stirring to form a first mixture, adding an organic acid to the first mixture to form a second mixture, and heating the second mixture to a first desired temperature. Upon reaching the first temperature, the second mixture is allowed to cool to a second desired temperature, and while cooling towards the second desired temperature, a synthetic polymer and then an acid are added to form a third mixture. The method further comprises the step of adding the first solution to the second solution at the second desired temperature,

The above-described and other features and advantages of the present invention will be appreciated and understood by those skilled in the art from the following detailed description, drawings, and appended claims.

### BRIEF DESCRIPTION OF DRAWINGS

FIG. 1 is a Table showing the Test Results of Study 1 for Sample A.

FIG. 2 is a Table showing the Test Results of Study 1 for Sample B;

FIG. 3 is a Chart comparing the Test Results for Sample A and Sample B for Study 1;

FIG. 4 is a Table showing the Test Results of Study 2;

FIG. 5 is a graph showing the Test Results of Study 2

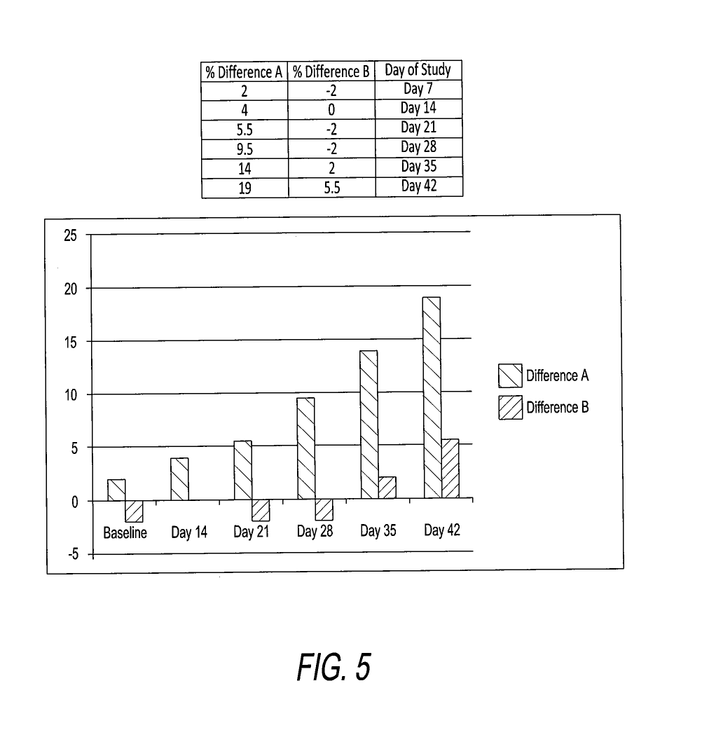

### DETAILED DESCRIPTION OF THE DISCLOSURE

The present disclosure provides leave-on, non-comedogenic and non-acnegenic (or non-acne-enhancing) hair cosmetic compositions, such as hair styling compositions. These compositions provide improved hair feel, for example suppleness and smoothness, and hair quality improving effects, for example improved manageability and luster. Advantageously, because the hair cosmetic compositions of the present disclosure are non-comedogenic and non-acnegenic, these compositions do not add to the blockage of pores, thereby reducing the incidence of pimples or blemishes.

The hair cosmetic compositions of the present disclosure contribute to essential quality improvement of the hair, and can provide excellent manageability and setting property to the hair. In addition, these compositions can impart the hair with improved flexibility touch and elasticity. More specifically, the hair cosmetic compositions of the present disclosure provide for proper setting of the hair and setting retention of a hairstyle, providing smoothness to the hair both during and after drying of the composition, and providing suppleness and smooth touch to the hair without stiffness.

When one or more antibacterial agents, an exfoliation agent, and an antioxidant are combined according to the present disclosure, the combination unexpectedly results in a non-comedogenic and non-acne-enhancing composition. The composition not only provides the above listed improved hair characteristics, but does not add to the clogging of skin pores. Without wishing to be bound to any particular theory, it is believed that the acidic pH of the cosmetic composition, and its lighter composition, acts in conjunction with the antibacterial properties and the antioxidant to contribute to the non-comedogenicity and non-acnegenicity of the cosmetic compositions. For example, it is believed that the exfoliation agent enables the antioxidant and antibacterial agent to penetrate the pores of the skin and thereby preventing clogging and exacerbation of acne.

For purposes of the present application, the term “non-comedogenic” is a term applied to cosmetic compositions that do not add to the blocking of pores. In theory, by not blocking pores, non-comedogenic products will not increase the incidence of pimples and blemishes. As such, the term “non-comedogenic” as defined herein, refers to cosmetic compositions that do not block pores and thereby do not increase the incidence of pimples.

Suitable antibacterial agents for the composition include, but are not limited to, microbiological preservatives and deodorant ingredients. In a preferred embodiment, a suitable antibacterial agent for use in the present disclosure is triclosan, which is, by way of example, sold under the trade name Irgasan PG 60.

The antibacterial agent is present in the cosmetic composition in an amount from 0.05% to 2.5%, and any subranges therebetween, based on the total weight of the composition. Preferably, the antibacterial agent is present in an amount from 0.05% to 1.5%, and any subranges therebetween, based on the total weight of the composition. An essential function of the antibacterial agent is suppression of acne-causing bacteria.

The exfoliating agent of the hair cosmetic composition can include several suitable compounds, for example, an acid. Suitable acids include, but are not limited to, lactic acid, citric acid, phosphoric acid, hydrochloric acid, salicylic acid, and glycolic acid. In a preferred embodiment, the exfoliating agent is glycolic acid, which can be sold under the trade name Glypure. The essential functions of the exfoliating agent are its ability to keep pores from clogging, enhance penetration of the antibacterial and antioxidants, lowering pH.

In one embodiment of the present disclosure, the exfoliating agent is present in the cosmetic composition in an amount from 0.1% to 10%, and any subranges therebetween, based on the total weight of the composition. Preferably, the exfoliating agent is present in an amount from 0.2% to 2.0%, and any subranges therebetween, based on the total weight of the composition.

The antioxidant of the hair cosmetic composition provides free radical suppression. Suitable antioxidants include, but are not limited, to ascorbic acid and various antioxidants listed in the CTFA dictionary. In an exemplary embodiment, a suitable antibacterial agent for use in the present disclosure is tocopheryl acetate which is, by way of example, sold under the trade name Tinoderm E. The antioxidant provides free radical suppression and decreases inflammation and the potential for inflammatory acne lesions.

In one embodiment of the present disclosure, the antioxidant is present in the cosmetic composition in an amount from 0.01% to 5.0%, and any subranges therebetween, based on the total weight of the composition. Preferably, the antioxidant is present in an amount from 0.05% to 3.0%, and any subranges therebetween, based on the total weight of the composition.

It is contemplated herein that the hair cosmetic compositions of the present disclosure may include a preservative. Suitable preservatives include one or more suitable organic acids, salts thereof, and synthetic preservatives. The organic acids are effective against yeast and mold. Suitable organic acids include, but are not limited to, sorbic acid, dehydroacetic acid, benzoic acid, and any salts thereof (e.g., sodium benzoate). Benzoic acid is, by way of example, sold as Benzoic Acid, USP. Suitable synthetic preservatives include, but are not limited to, methylisothiazolinone and parabens.

In one embodiment of the present disclosure, the preservative is present in the cosmetic composition in an amount from 0.01% to 5.0%, and any subranges therebetween, based on the total weight of the composition. Preferably, the preservative is present in an amount from 0.1% to 1.0%, and any subranges therebetween, based on the total weight of the composition.

It is contemplated herein that the hair cosmetic compositions of the present disclosure also include one or more cationic polymers. The cationic polymers functions primarily to assist in holding the hair, thickening the hair, and aiding in the combing of hair. Suitable cationic polymers include, but are not limited to polyquaternium-4, polyquaternium-10, polyquaternium-7, and polyquaternium-11. Polyquaternium-4 is, by way of example, sold under the trade name Celquat H-100.

In one embodiment of the present disclosure, cationic polymer is present in the cosmetic composition in an amount 0.1% to 7.0%, and any subranges therebetween, based on the total weight of the composition. Preferably, the cationic polymer is present in an amount from 0.5% to 3.0%, and any subranges therebetween, based on the total weight of the composition.

It is contemplated herein that the hair cosmetic compositions of the present disclosure also include one or more synthetic polymers for the primary purpose of thickening the formulation. The synthetic polymers also assist in holding the hair, thickening the hair, and aiding in the combing of the hair. Suitable synthetic polymers include various cellulosic-derivatives including but not limited too hydroxyethylcellulose and hydroxypropylcellulose, and derivatives thereof. In addition, it is contemplated herein that synthetic polymers may also include various viscosity increasing agents listed in the CTFA Dictionary. Acrylates/ammonium methacrylate copolymer is, by way of example, sold under the trade name Structure Plus (20%).

In one embodiment of the present disclosure, the synthetic polymer is present in the cosmetic composition in an amount from 0.5% to 20.0%, and any subranges therebetween, based on the total weight of the composition. Preferably, the synthetic polymer is present in an amount from 3.0% to 15.0%, and any subranges therebetween, based on the total weight of the composition.

It is contemplated herein that the hair cosmetic compositions of the present disclosure may also include one or more additional synthetic polymers that function primarily as hair fixative, hair holding, bodifying, and thickening agents. Suitable additional synthetic polymers include, but are not limited to the various synthetic polymers listed under hair fixative in the CTFA Dictionary. One suitable synthetic polymer is polyvinylpyrrolidone/vinyl acetate (VP/VA) copolymer which is, by way of example, sold under the trade name PVP/VA W-735.

In one embodiment of the present disclosure, the additional synthetic polymer is present in the cosmetic composition in an amount from 0.1% to 5.0%, and any subranges therebetween, based on the total weight of the composition. Preferably, the additional synthetic polymer is present in an amount from 0.3% to 2.5%, and any subranges therebetween, based on the total weight of the composition.

It is contemplated herein that the hair cosmetic compositions of the present disclosure may also include one or more nonionic surfactants. The nonionic surfactants function as solubilizers. Suitable nonionic surfactants include, but are not limited to, the various alkoxylated alcohols listed in the CTFA Dictionary. One suitable nonionic surfactant is octoxynol-9 which is, by way of example, sold under the trade name Triton X-100.

In one embodiment of the present disclosure, the nonionic surfactant is present in the cosmetic composition in an amount from 0.1% to 5.0%, and any subranges therebetween, based on the total weight of the composition. Preferably, the nonionic surfactant is present in an amount from 0.2% to 2.5%, and any subranges therebetween, based on the total weight of the composition.

It is contemplated herein that the hair cosmetic compositions of the present disclosure may also include a fragrance. A fragrance adds to the aesthetic appeal and support product functionality of the cosmetic composition. Suitable fragrances include, in one embodiment, a fragrance sold under the trade name SM 85835M.

In one embodiment of the present disclosure, the fragrance is present in the cosmetic composition in an amount from 0.05% to 2.5%, and any subranges therebetween, based on the total weight of the composition. Preferably, the fragrance is present in an amount from 0.1% to 1.5%, and any subranges therebetween, based on the total weight of the composition.

It is contemplated herein that the hair cosmetic compositions of the present disclosure may also include one or more silicone fluids. Silicone fluids impart a smooth feel and shine to the hair. In addition, the silicone fluid adds to the ease in which hair may be combed. Suitable silicone fluids include, but are not limited to, dimethicones, dimethicone polyols, and dimethicone esters. For example, in one embodiment, the cosmetic composition includes PEG-12 dimethicone, sold under the trade name Dow Corning 193 Fluid.

In one embodiment of the present disclosure, the silicone fluid is present in the cosmetic composition in an amount from 0.05% to 5.0%, and any subranges therebetween, based on the total weight of the composition. Preferably, the silicone fluid is present in an amount from 0.05% to 2.5%, and any subranges therebetween, based on the total weight of the composition.

It is contemplated herein that the hair cosmetic compositions of the present disclosure may also include one or more modified phospholipids. The modified phospholipids play a role primarily in conditioning the hair, co-solubilizing formula ingredients and plasticizing hair holding/setting polymers. Suitable modified phospholipids for use in the cosmetic compositions of the present disclosure include, but are not limited to, alkanolamides, amphoteric surfactants, and alkanolamines. By way of example, one suitable modified phospholipid is sodium coco PG-dimonium chloride which is, by way of example, sold under the trade name Phospholipid CDM.

In one embodiment of the present disclosure, the modified phospholipid is present in the cosmetic composition in an amount 0.1% to 7.5%, and any subranges therebetween, based on the total weight of the composition. Preferably, the modified phospholipid is present in an amount from 0.5% to 4.0%, and any subranges therebetween, based on the total weight of the composition.

In addition, the present disclosure contemplates that the hair cosmetic compositions may include one or more quaternary ammonium compounds. The quaternary ammonium compounds function to ease combing, static flyaway suppression, manageability, and general conditioning of the hair. There are several quaternary ammonium compounds suitable for use in the cosmetic compositions including, but not limited to, quaternary ammonium chloride and hair conditioning agents suitable for hair care use as listed in the CTFA Dictionary. By way of example, one suitable quaternary ammonium compound is cetrimonium chloride which is, by way of example, sold under the trade name Arquad 16-29 W (29%).

In one embodiment of the present disclosure, the quaternary ammonium compound is present in the cosmetic composition in an amount from 0.1% to 6.0%, and any subranges therebetween, based on the total weight of the composition. Preferably, the quaternary ammonium compound is present in an amount from 0.3% to 3.0%, and any subranges therebetween, based on the total weight of the composition.

Each of the hair cosmetic compositions will also include water which serves as a diluent and solvent. Water includes deionized water.

The following is an example of the present disclosure. Unless otherwise indicated below, all parts, percentages, and proportions are by weight.

### Example 1

| 1. | Ingredient-Trade Name | Ingredient-INCI Name | % wt |
|---|---|---|---|
| 2. | Deionized water | Water | 81.66 |
| 3. | Celquat H-100 | Polyquaternium-4 | 1.00 |
| 4. | Benzoic Acid, USP | Benzoic Acid | 0.20 |
| 5. | Structure plus (20%) | Acrylates/Ammonium | 12.50 |
| | | Methacrylate Copolymer | |
| 6. | Glycolic Acid, USP (70%) | Glycolic Acid | 0.63 |
| 7. | PVP/VA W-735 | VP/VA Copolymer | 1.35 |
| 8. | Triton X-100 | Octoxynol-9 | 0.54 |
| 9. | SM 85835M | Fragrance | 0.54 |
| 10. | Dow Corning 193 Fluid | PEG-12 Dimethicone | 0.10 |
| 11. | Phospholipid CDM | Sodium Coco PG- | 0.50 |
| | | Dimonium Chloride | |
| | | Phosphate | |
| 12. | Irgasan PG 60 | Triclosan | 0.06 |
| 13. | Tinoderm E | Tocopheryl Acetate | 0.06 |
| 14. | Arquad 16-29 W (29%) | Cetrimonium Chloride | 0.86 |
| Total | | | 100.00 |

Procedure: The following is a procedure for making an embodiment of a hair cosmetic composition according to the present disclosure:

### Part A

Combine at room temperature the following components in the following order:

a. Triton X-100

b. SM 85835M

c. DC-193

d. Phospholipid CDM

e. Irgasan PG

f. Tocopherol Acetate

g. Arquad 16-29 (29%)

While the above listed order of ingredients is preferred, some modification to the order is acceptable.

### Part B

1. To the deionized water, add Celquat H-100 with moderate to fast stirring.
2. To the deionized water and Celquat H-100 mixture, add the Benzoic Acid with moderate to fast stirring.
3. When the deionized water/Celquat H-100/Benzoic Acid is uniformly suspended, heat the mixture to 50 degrees Celsius.
4. Stir the mixture until clear and uniform (approximately 15 minutes).
5. Cool the mixture to approximately 30 to 35 degrees Celsius.
6. When the mixture reaches 30 to 35 degrees Celsius, add in order, Structure Plus, Glycolic Acid, and the PVP/VA W-735.

The synthetic polymer (Structure Plus), acid (glycolic acid), and additional synthetic polymer (PVP/VA W-735) can be added at any point when the mixture of Part B is cooling, but the higher the temperature that these three components are added, the better.

### Part C

Add the mixture from Part A to the mixture from Part B at 25 to 35 degrees Celsius.

Evaluation Criteria

Study 1: Feasibility study to compare the comedogenic and acnegenic potential of two cosmetic products in use conditions

Objective: Certain hair styling products have the ability to cause acne type reactions in the hairline region on subjects with pre-inclination to this condition. The study evaluated and compared the comedogenic and acnegenic potential of two topically applied hair styling products over six weeks of daily use.

Test Sample Description: Sample A is a commercially available cosmetic composition having a formulation that is outside the scope of the present disclosure. The actual formulation is not available, but the listing of ingredients provided on the product package is as follows: water, PVP, sorbitol, carbomer, keratin amino acids, panthenyl ethyl ether, palmitoyl oligopeptide, glyceryl polymethacrylate, fragrance, glycerin, propylene glycol, oleth 20, aminomethyl propanol, rahnella/soy protein ferment, PEG 8, potassium sorbate, tetrasodium EDTA, methylparaben, diazolidinyl urea, methylchloroisothiazolinone, methylisothiazolinone, benzophenone-4, Orange 4, and Yellow 5. Sample B is a cosmetic hair gel composition in accordance with the present disclosure and having the formulation of Example 1 above.

Standards for inclusion in the Study included: utilization of Individuals eighteen years of age or older; Individuals exhibiting inflammatory and/or noninflammatory acne lesions in hairline region; and individuals with no known abnormal response or history of hypersensitivity to cosmetics in general and hair products in particular.

The Study utilized thirteen subjects exhibiting Grade 1 or greater acne condition in the hairline region of the temple and forehead were inducted into this study. Prior to study commencement, each panelist was examined and qualified by the Study Director.

Panelists were required to abstain from using any other topical and/or oral treatment for the duration of the study. Subjects were supplied with the test materials of Sample A and Sample B and were instructed to apply them once a day to clean hair on a split-head basis, Sample A was applied to one side of the head and Sample B was applied to the other throughout the study. On the initial day of study and again after 7, 14, 21, 28, 35 and 42 days of daily product application visual evaluations, grading of acne and skin irritation, counts of inflammatory and/or noninflammatory acne lesions (open and closed comedones, papules and pustules) were conducted by the study Director. Lab Scale digital photography was obtained at each time point for comparison.

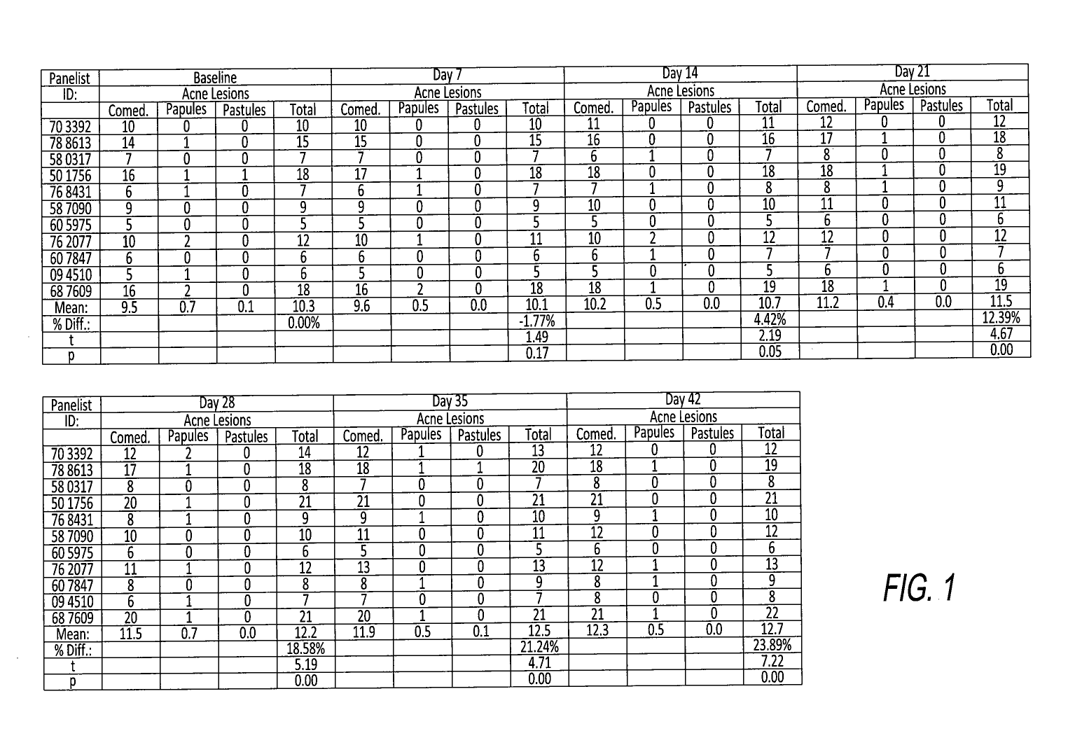

Results: FIG. 1 is a Table showing the Test Results for Sample A (a hair cosmetic composition having a formula outside the scope of the present application). As shown, on Day 1 of the Study, the baseline mean value of acne lesions of the test subjects was 10.3. On Day 7, the mean value of acne lesions was 10.1, a 1.77% decrease. On Day 14, the mean value of acne lesions was 10.7, a 4.42% increase from the baseline. On Day 21, the mean value was 12.39%, a 4.67% increase from the base line. On Day 28, the mean value of acne lesions was 12.2, a 18.58% increase from the base line value. On Day 35, the mean value of acne lesions was 12.5, a 21.24% increase from the baseline value. On Day 42, the mean value of acne lesions was 12.7, a 23.89% increase from the baseline value.

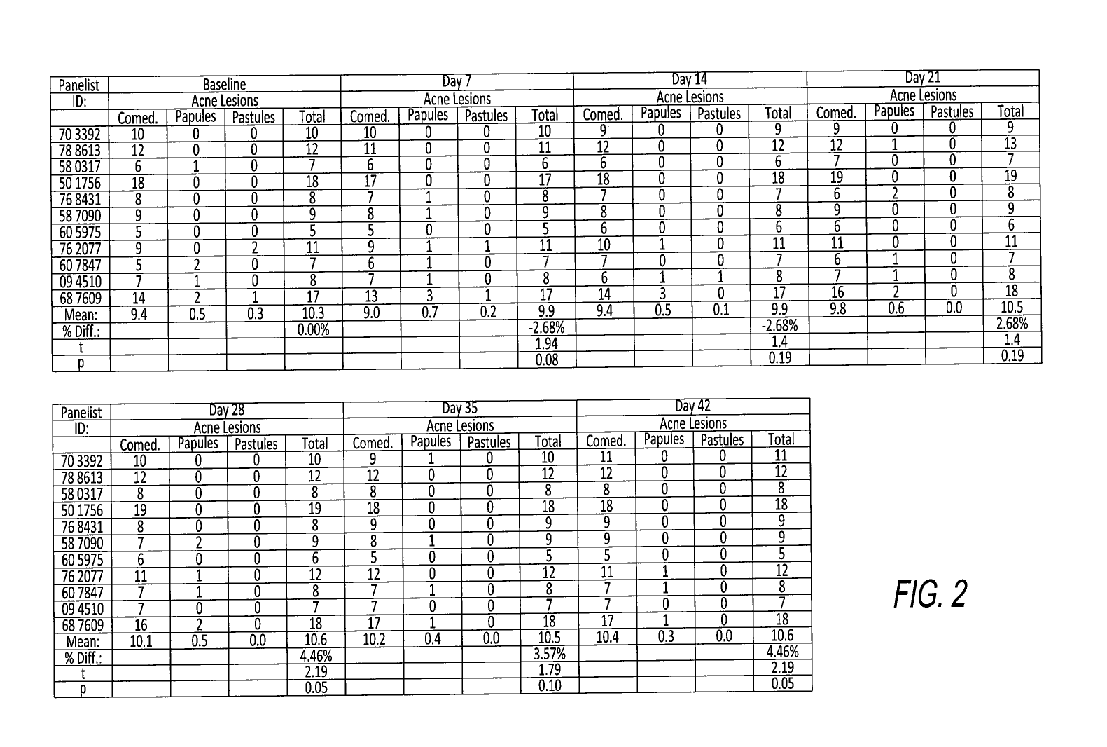

FIG. 2 is a Table showing the Test Results for Sample B. As shown, on Day 1 of the Study, the baseline mean value of acne lesions of the test subjects was 10.2. On Days 7 and 14, mean value of acne lesions was 9.9, a 2.68% decrease. On Day 21, the mean value of acne lesions was 10.5, a 2.68% increase from the baseline. On Day 28, the mean value was 10.6%, a 4.46% increase from the baseline. On Day 35, the mean value of acne lesions was 10.5, a 3.57% increase from the baseline value. On Day 42, the mean value of acne lesions was 10.6, a 4.46% increase from the baseline value.

Thus, over the course of the 42 day study, Sample A showed an increase in the mean value of acne lesions of 23.89%. Sample B, however, displayed an increase in the mean value of acne lesions of only 4.46%.

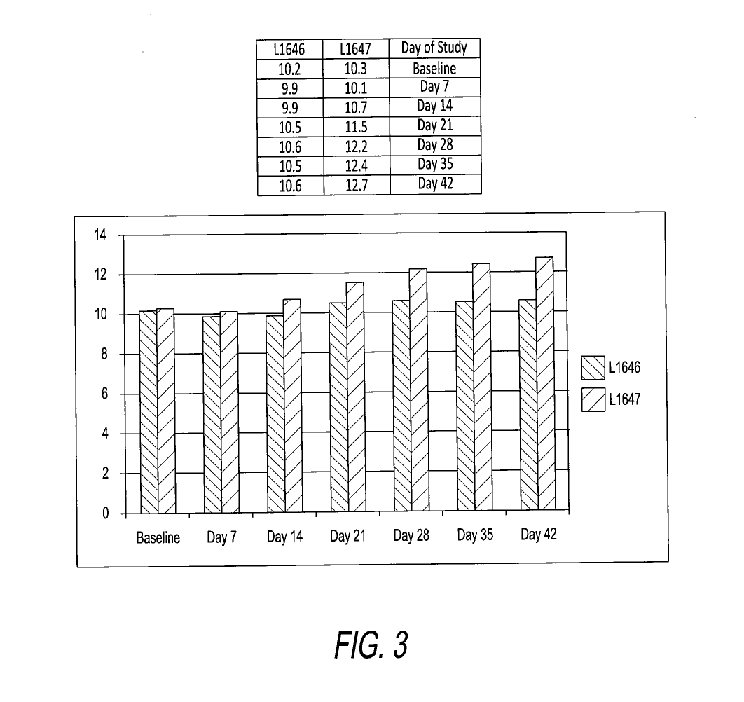

FIG. 3 shows the test results of Study 1, in which Total Acne Lesion Counts are plotted versus Differences in Acne Lesion Counts. As shown, Sample B shows a much smaller increase in the number of acne lesions over the course of the study than that for Sample A.

No adverse effects or unexpected reactions of any kind were observed on any subject.

The following conclusions were drawn:

When applied on a split-head basis to a panel of subjects pre-inclined to acne, test material Sample A demonstrated statistically significant increases in hairline area acne lesion counts observed from the third through the sixth week, namely day 22 through day 42, of use. Application of Sample B demonstrated no statistically significant differences in hairline acne lesion counts.

Study 2: Feasibility study to compare the comedogenic potential of two cosmetic products under in use conditions

Objective: Certain hair styling products or basic raw materials have the ability to cause acne type reactions in the hairline region on subjects with pre-inclination to this condition. It is the intention of this study to evaluate and compare the comedogenic potential of two topically applied hair styling products over six weeks of daily use.

Test Sample Description:

Sample A is the commercially available cosmetic composition having a formulation that is outside the scope of the present disclosure, discussed above in Study 1. Sample B is a cosmetic hair gel composition in accordance with the present disclosure and having the formulation shown in Example 1 above.

Standards for inclusion in a Study included: utilization of Individuals eighteen years of age or older; Individuals exhibiting inflammatory and/or noninflammatory acne lesions in hairline region; and Individuals with no known abnormal response to cosmetic products and who are willing to cooperate with the study requirements.

The Study utilized three subjects exhibiting Grade 1 or greater acne condition in the hairline region of the temple and forehead. Panelists were required to abstain from using any other topical and/or oral treatment for the duration of the study. Subjects were supplied with the test materials of Sample A and Sample B and were instructed to apply them once a day to clean hair on a split-head basis wherein Sample A was applied to one side of the head and Sample B was applied to the other throughout the Study.

On the initial day of Study and again after 7, 14, 21, 28, 35 and 42 days of daily product application visual evaluations, grades of acne and skin irritation, counts of inflammatory and/or noninflammatory acne lesions (open and closed comedones, papules and postules) were conducted by the Study Director. Lab Scale digital photography was obtained at each time point for comparison.

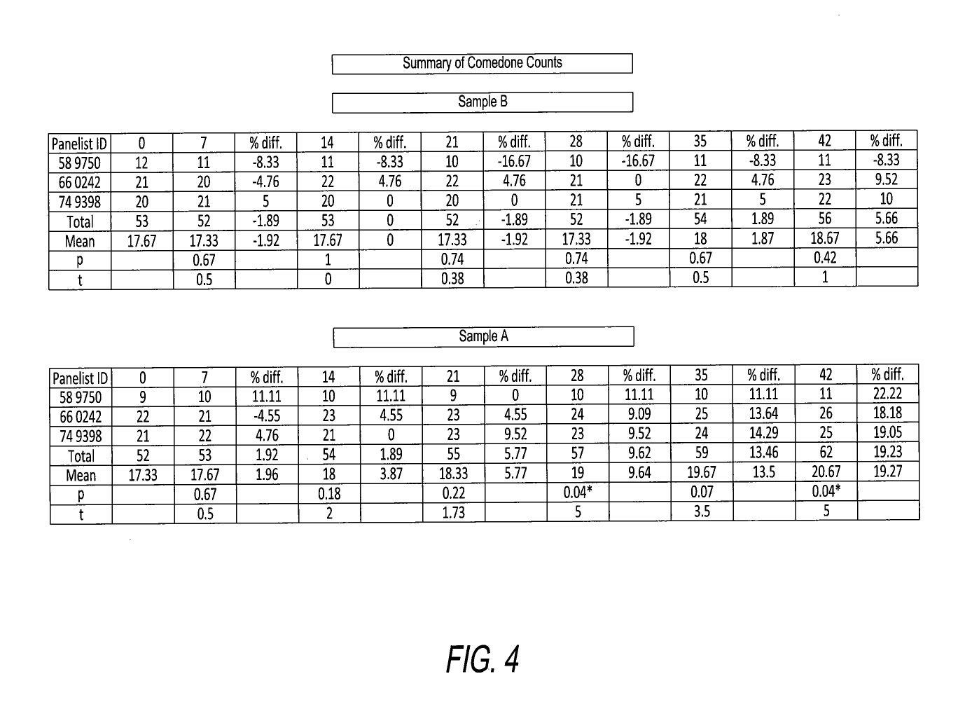

Results: FIG. 4 is a Table showing the results of Study 2. As can be seen, over the course of the 42 day study application of Sample A resulted in a 19.23% increase in the mean value of comedone counts. By contrast, use of Sample B resulted in only a 5.66% increase in the mean value of comedone counts.

No adverse effects or unexpected reactions of any kind were observed on any subject.

The following conclusions were drawn:

Topical application of the test material Sample A demonstrated statistically significant increases in hairline area comedone counts observed after four and six weeks of use in subjects with pre-inclination to this condition. Sample B by comparison demonstrated no statistically significant differences in hairline comedone counts when applied on a split-head basis to the same test panel.

As seen from the test data above, the use of an embodiment of a hair cosmetic composition having a formulation in accordance with the present disclosure resulted in a statistically insignificant increase in the occurrence of acne lesions and comedones. The reduction in the occurrence of acne lesions and comedones when compared to the results of testing of a market leading commercially available product attests to the noncomedogenic and nonacnegenic nature of the present composition.

While the present disclosure has been described with reference to one or more exemplary embodiments, it will be understood by those skilled in the art that various changes may be made and equivalents may be substituted for elements thereof without departing from the scope of the present disclosure. In addition, many modifications may be made to adapt a particular situation or material to the teachings of the disclosure without departing from the scope thereof. Therefore, it is intended that the present disclosure not be limited to the particular embodiment(s) disclosed as the best mode contemplated, but that the disclosure will include all embodiments falling within the scope of the appended claims.

1. A hair cosmetic composition, comprising:
an exfoliating agent in an amount of 0.1% to 10%, based on the total weight of the hair cosmetic composition;
an antioxidant in an amount of 0.01% to 5.0%, based on the total weight of the hair cosmetic composition; and
an antibacterial agent in an amount of 0.05% to 2.5%, based on the total weight of the hair cosmetic composition,
wherein the hair cosmetic composition is noncomedogenic.

2. The hair cosmetic composition of claim 1, wherein said exfoliating agent is an acid selected from the group consisting of glycolic acid, lactic acid, citric acid, and salicylic acid.

3. The hair cosmetic composition of claim 1, wherein said exfoliating agent is present in an amount of 0.2% to 2%, based on the total weight of the hair cosmetic composition.

4. The hair cosmetic composition of claim 1, wherein said antioxidant is tocopheryl acetate.

5. The hair cosmetic composition of claim 4, wherein said antioxidant is present in an amount of 0.05% to 3.0%, based on the total weight of the hair cosmetic composition.

6. The hair cosmetic composition of claim 1, wherein said antibacterial agent is selected from the group consisting of a microbiological preservatives and a deodorant ingredient.

7. The hair cosmetic composition of claim 1, wherein said antibacterial agent is tricolsan.

8. The hair cosmetic composition of claim 1, wherein said antibacterial agent is present in an amount of 0.05% to 1.5%, based on the total weight of the hair cosmetic composition.

9. The hair cosmetic composition of claim 1, further comprising a cationic polymer.

10. The hair cosmetic composition of claim 9, wherein said cationic polymer is present in an amount of 0.1% to 7.0%, based on the total weight of the hair cosmetic composition.

11. The hair cosmetic composition of claim 9, wherein said cationic polymer is present in an amount of 0.5% to 3.0%, based on the total weight of the hair cosmetic composition.

12. The hair cosmetic composition of claim 1, further comprising a preservative.

13. The hair cosmetic composition of claim 12, wherein said preservative is an organic acid.

14. The hair cosmetic composition of claim 13, wherein the organic acid is selected from the group consisting of sorbic acid, sorbic acid salt, dehydroacetic acid, dehydroacetic acid salt, benzoic acid, benzoic acid salt, and any combination thereof.

15. The hair cosmetic composition of claim 12, wherein said preservative is a synthetic preservative selected from the group consisting of methylisothiazolinone and a paraben.

16. The hair cosmetic composition of claim 12, wherein said preservative is present in an amount of 0.01% to 5.0%, based on the total weight of the hair cosmetic composition.

17. The hair cosmetic composition of claim 12, wherein said preservative is present in an amount of 0.1% to 1.0%, based on the total weight of the hair cosmetic composition.

18. The hair cosmetic composition of claim 1, further comprising at least one component selected from the group consisting of a synthetic polymer, a nonionic surfactant, silicone, a modified phospholipid, and ammonium chloride.

19-21. (canceled)
